(19) United States
(12) Patent Application Publication
(10) Pub. No.: US 2025/0369004 A1
(43) Pub. Date: Dec. 4, 2025

(54) GIBBERELLIC ACID AND THE FORMATION OF MALE REPRODUCTIVE STRUCTURES ON FEMALE CANNABIS PLANTS

(71) Applicant: Rev Genomics, Inc., Oakland, CA (US)

(72) Inventor: Bertrand R. Vick, Oakland, CA (US)

(73) Assignee: Rev Genomics, Inc., Oakland, CA (US)

(21) Appl. No.: 19/225,975

(22) Filed: Jun. 2, 2025

Related U.S. Application Data
(60) Provisional application No. 63/654,846, filed on May 31, 2024.

(51) Int. Cl. C12N 15/82; C12N 9/22; C12N 15/11

(52) U.S. Cl. CPC C12N 15/8243; C12N 9/226; C12N 15/111; C12N 15/8206; C12N 15/8213; C12N 2310/20

(57) ABSTRACT

Exemplary embodiments described herein include cannabis females that have a potential to form male reproductive parts that may be treated in a manner to affect the gibberellic acid pathway to prevent the formation of male reproductive parts. Additionally, cannabis females may be treated with exogenous compounds (sprays, chemicals, etc.) to reduce the amount of gibberellic acid produced.

### CROSS REFERENCE TO RELATED PATENT APPLICATIONS

The present application claims priority benefit, under 35 U.S.C. § 119 (e), to co-pending and commonly-assigned U.S. Provisional Patent Application No. 63/654,846, filed May 31, 2024, entitled “DISRUPTION OF THE GIBBERELLIC ACID PATHWAY ON PLANT GROWTH AND DEVELOPMENT TO INHIBIT THE FORMATION OF MALE REPRODUCTIVE STRUCTURES ON FEMALE CANNABIS PLANTS”, and listing as inventors Bertrand R. Vick and Phillip Adam Conklin, which application is herein incorporated by reference as to its entire content.

### BACKGROUND

One skilled in the art will recognize the increasing growth and applicability of cannabis-based products within today's society and health markets. For example, the use of medical cannabis has been known to treat a variety of medical conditions including anxiety, sleep disorder and other types of conditions. This expanding use of medical cannabis products has resulted in a variety of treatments that are oftentimes more effective with less side-effects than previously used traditional medicines. In addition, recreational cannabis use is drastically increasing as state laws have become more tolerant in its use. This increasing demand has led to a need to more efficiently and dynamically develop growth techniques within the cannabis markets.

Cannabis plants may be differentiated across three types: namely female cannabis, male cannabis and hermaphrodite cannabis. In most instances, the female cannabis plant is preferred across a variety of different cannabis uses and applications. However, maximizing female cannabis plants across a cannabis crop presents certain challenges and failure to address these challenges may result in a meaningful amount of male or hermaphrodite cannabis plants being present. One factor in the value of a cannabis crop is the ratio of female cannabis plants to male/hermaphrodite plants in that a higher density of female cannabis results in an increase in value and applicability across different uses.

Accordingly, it is desired that systems and methods be provided that increase the amount of female cannabis plants within a crop.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 is a schematic diagram of an exemplary gibberellic acid pathway according to various embodiments of the invention.

FIG. 2 is a schematic diagram of an alternative exemplary gibberellic acid pathway according to various embodiments of the invention.

FIG. 3 is a schematic diagram of exemplary steps for performing mutagenesis according to various embodiments of the invention.

FIG. 4 is a schematic diagram of exemplary steps for performing CRISPR-Cas9 according to various embodiments of the invention.

### DETAILED DESCRIPTION

Exemplary embodiments described herein include cannabis females that have a potential to form male reproductive parts that may be treated in a manner to affect the gibberellic acid pathway to prevent the formation of male reproductive parts. Additionally, cannabis females may be treated with exogenous compounds (sprays, chemicals, etc.) to reduce the amount of gibberellic acid produced. Examples of chemicals or sprays that could be used to disrupt or interact with GA pathway(s) include, however not limited to, paclobutrazol, jasmonic acid/jasmonate, ethephon and/or ethylene and other chemicals known to one of skill in the art.

Cannabis females may also be treated with exogenous compounds (sprays, chemicals, etc.) to reduce the effects of gibberellic acid on the biology of the plant. Examples of chemicals or sprays that could be used to disrupt or interact with GA pathway(s) include, however not limited to, paclobutrazol, jasmonic acid/jasmonate, ethephon and/or ethylene and other chemicals known to one of skill in the art. Further exemplary embodiments include the biology of the plant being altered, through mutagenesis, CRISPR, or other molecular genetics tools, to produce less gibberellic acid. Additionally, the biology of the plant may be altered, through mutagenesis, CRISPR, or other molecular genetics tools, to be less sensitive to gibberellic acid.

Further exemplary embodiments include environmental factors being manipulated to cause the plant to produce less gibberellic acid and/or environmental factors being manipulated to cause the plant to be less sensitive to gibberellic acid. Examples of environmental manipulations that change GA biosynthesis include, however not limited to, manipulating light levels (higher light causes higher GA levels).

Cannabis (both hemp and “marijuana”) is typically dioecious, meaning that male and female reproductive organs exist on separate individuals (although there are rare exceptions to this rule). Thus, when cannabis seeds are created by crossing a male to a female, statistically half the seeds will create male plants, and half the seeds will create female plants, which is undesirable for much cannabis use cases, as it is primarily the female plants that produce valuable secondary metabolites (THC, CBD, etc.). It is possible to produce “feminized” seeds that will give rise to mostly female plants by coaxing, using primarily chemical methods, a female cannabis plant to produce male reproductive parts and pollen. Since cannabis uses an X-Y sex-determination system like humans, genetically female plants can make XX pollen using chemical feminization methods. When XX pollen is used to fertilize a female cannabis plant, all of the offspring will be female.

However, cannabis seeds, especially feminized seeds, may give rise to female plants that have the unfortunate characteristic of forming male reproductive parts (often called ‘hermaphrodites’). In a commercial setting, female plants with male reproductive parts could ruin a crop by pollinating other females in the field, greenhouse, or controlled environment (pollinated cannabis plants produce fewer secondary metabolites, and cannabis end consumers do not like their flower products to contain seeds). It has long been thought that when hermaphrodites occur, it is due to an unavoidable genetic trait or is somehow an epigenetic result of the chemicals used to reverse the females to make the feminized pollen.

The formation of male parts on female cannabis plants may be a side effect of high amounts of gibberellic acid being created by especially vigorous plants. Therefore, it would be possible to prevent the undesirable formation of these male parts by doing one or more of the following: influence the plant to produce less gibberellic acid or disrupt the effects that gibberellic acid exerts on the plant.

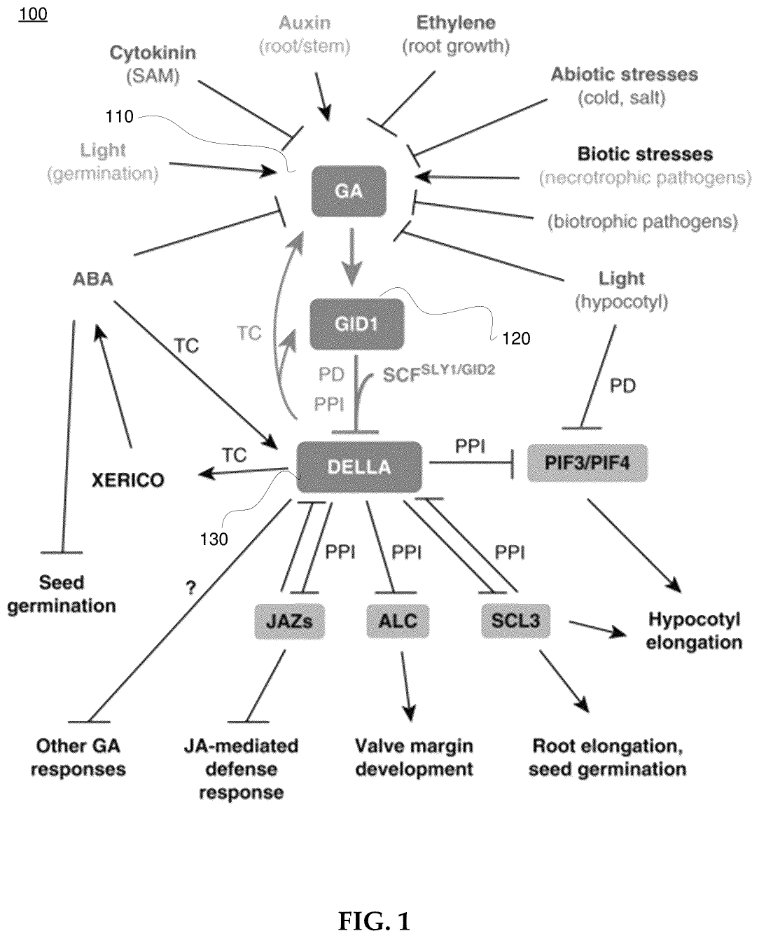

FIG. 1 is a schematic diagram of an exemplary gibberellic acid pathway according to various embodiments of the invention.

According to various exemplary embodiments, bioactive gibberellins (GAS) 110 are diterpene phytohormones that modulate growth and development throughout the whole life cycle of the flowering plant. Impressive advances have been made in elucidating the GA pathway with the cloning and characterization of genes encoding most GA biosynthesis and catabolismenzymes, GA receptors (GIBBERELLIN INSENSITIVE DWARF1, GID1) 120 and early GA signaling components. Recent biochemical, genetic and structural analyses demonstrate that GA de-represses its signaling pathway by GID1-induced degradation of DELLA proteins 130, which are master growth repressors, via a ubiquitin-proteasome pathway. Multiple endogenous signals and environmental cues also interact with the GA-GID1-DELLA regulatory module by affecting the expression of GA metabolism genes, and hence GA content and DELLA levels. Importantly, DELLA 130 integrates different signaling activities by direct protein-protein interaction with multiple key regulatory proteins from other pathways.

The functional GA-GID1-DELLA module 100 is highly conserved among vascular plants, but not in the bryophytes. Differentiation of the moss Physcomitrella patens is regulated by unidentified ent-kaurene-derived diterpenes, which are distinct from the common active GAs in vascular plants.

Gibberellins (GAs) exert their control over plant growth and development mainly through the GA-GID1-DELLA signaling module. This module involves the hormone GA, its receptor GID1, and the transcriptional repressors DELLA proteins. GID1, a soluble receptor, binds to GA, and this interaction triggers the degradation of DELLA proteins, thereby lifting their repression of GA signaling. This process ultimately leads to plant growth and development, and excessive activation of this module, through certain artificial light regimes utilized in the cannabis industry, leads to excessive GA signaling and hermaphrodites. One skilled in the art will recognize that ethylene can be inhibitory of GA signaling and therefore suppressive of hermaphrodites.

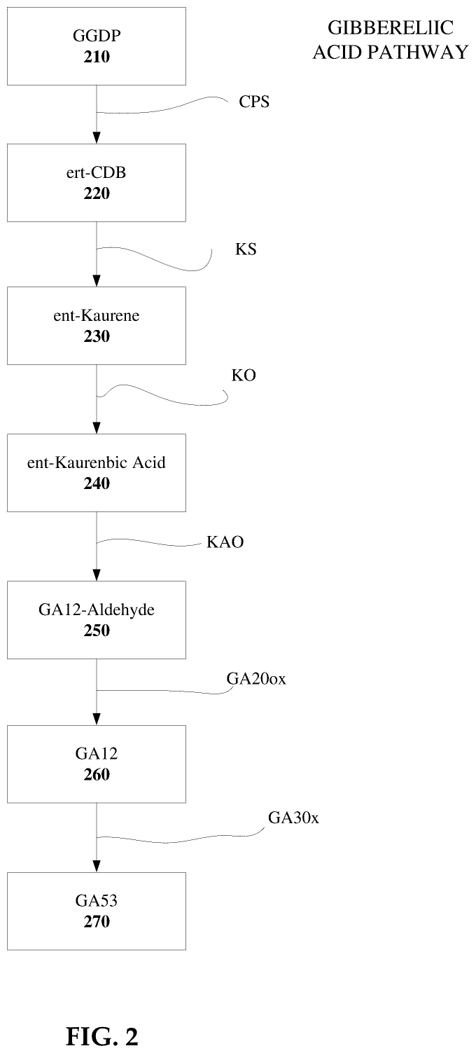

FIG. 2 is a schematic diagram of an alternative exemplary gibberellic acid pathway according to various embodiments of the invention.

The gibberellic acid (GA) biosynthesis pathway involves several key enzymatic steps, each contributing to the formation of various intermediates and ultimately producing active gibberellins. A detailed description of each reaction follows:

GGDP (Geranylgeranyl diphosphate) 210 is converted to ent-CDP (ent-Copalyl diphosphate) 220 using a first enzyme such as ent-Copalyl diphosphate synthase (CPS) according to various embodiments of the invention. Specifically, GGDP 210 undergoes cyclization to form ent-CDP 220 in response to the application of the CPS enzyme. This reaction involves the formation of a diterpene skeleton through a series of rearrangements and cyclizations. One skilled in the art will recognize that other enzymes may be used to produce ent-CDP from GGDP substances.

The resulting ent-CDP 220 is converted to ent-Kaurene 230 using a second enzyme such as ent-Kaurene synthase (KS) according to various embodiments of the invention. Specifically, ent-CDP 220 is converted to ent-Kaurene 230 via another cyclization process in response to the applications of the KS enzyme. This step completes the formation of the tetracyclic diterpenoid structure characteristic of gibberellins. One skilled in the art will recognize that other enzymes may be used to produceent-Kaurene from ent-CDP substances.

Ent-Kaurene 230 is converted to ent-Kaurenoic Acid 240 using a third enzyme such as ent-Kaurene oxidase (KO) according to various embodiments of the invention. Specifically, ent-Kaurene 230 undergoes a series of oxidations, converting it to ent-Kaurenoic acid 240. In certain examples, this process may involve the introduction of oxygen atoms to the molecule, facilitating further modifications. One skilled in the art will recognize that other enzymes may be used to convert ent-Kaurenoic acid from ent-Kaurene.

Ent-Kaurenoic Acid 240 is converted to GA12-Aldehyde 250 using a fourth enzyme such as ent-Kaurenoic acid oxidase (KAO) according to various embodiments of the invention. Specifically, ent-Kaurenoic acid 240 is oxidized to form GA12-Aldehyde 250. In certain examples, this reaction involves the removal of carboxyl groups and the introduction of aldehyde functional groups. One skilled in the art will recognize that other enzymes may be used to produce GA12-Aldhyde from ent-Kaurenoic acid.

GA12-Aldehyde 250 is converted to GA12 260 using a fifth enzyme such as GA 20-oxidase (GA20ox) according to various embodiments of the invention. Specifically, GA12-Aldehyde 250 is further oxidized to form GA12 260 in response to the application of the GA20ox. In this example, GA12 is generated by the oxidation of the aldehyde group to a carboxylic acid group. One skilled in the art will recognize that other enzymes may be used to generate GA12 from GA12-Aldehyde.

GA12 260 is converted to GA53 270 using a sixth enzyme such as GA 3-oxidase (GA3ox) according to various embodiments of the invention. Specifically, GA12 260 undergoes a hydroxylation reaction to form GA53 270 in response to the application of GA3ox. In this example, additional hydroxyl groups are introduced into the GA12 molecule resulting in an increase to the complexity and biological activity of the molecule.

One skilled in the art will understand that these enzymatic steps represent the core pathway for the biosynthesis of gibberellic acids, which are important plant hormones regulating growth and development. The final products, active gibberellins like GA1 and GA3, are derived from GA53 through further modifications, including hydroxylation and oxidation reactions in certain embodiments of the invention.

The metabolites are shown in boxes within FIG. 2 as well as the enzymes responsible for each conversion are shown between the boxes. In certain embodiments, the various enzymes would be upregulated or downregulated, or completely ablated, with mutagenesis or CRISPR techniques, leading to effects on GA biosynthesis and therefore hermaphrodites. For instance, GGDP=geranylgeranyl diphosphate (metabolite). KS=ent-kaurene synthase (enzyme with corresponding gene(s)).

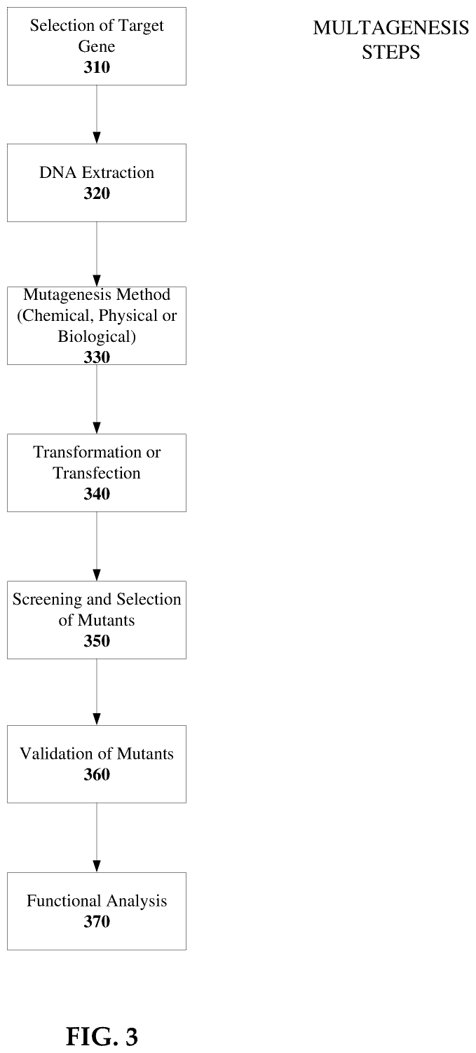

FIG. 3 is a schematic diagram of exemplary steps for performing mutagenesis according to various embodiments of the invention. As shown, the first step relates to the selection of a target gene 310 in which a specific gene or genes of interest is identified. In certain examples, the selected target gene should have a known or hypothesized function that is relevant to the mutagenesis process. This function helps enable that mutations introduced during this process will yield into the relevant gene function or relevant biological process. This step also isolates a mutagenesis target (e.g., embryo, cell line, etc.).

In a next step, DNA is extracted 320 from an organism for further processing according to various embodiments of the invention. Extracting DNA from the organism may be essential for downstream applications because the integrity and purity of DNA may affect the efficiency and accuracy of the mutagenesis process. In certain embodiments, this step treats a target with one or more mutagens (e.g., EMS (ethyl methane sulfonate), etc.) In certain instances, contaminants or degraded DNA can lead to errors or reduce the success rate of introducing mutations.

After DNA extraction, a mutagenesis method is identified 330 based at least in part on the DNA according to various embodiments of the invention. In certain examples, this mutagenesis method may be chemical, physical, biological or a combination thereof. Oftentimes, the identification of the mutagenesis method correlates to the generation of desired mutations. For example, chemical mutagens (e.g., EMS) may introduce random mutations by altering DNA bases while physical methods (e.g., UV radiation) may cause DNA damage that leads to mutations during repair. Biological methods (e.g., CRISPR-Cas9) may offer precise, targeted modifications. In certain embodiments, this step effectively screens for mutations in target gene(s) (e.g., TILLING (targeting induced local lesions in genomes). One skilled in the art will recognize that the method chosen may depend on the study's goals, such as creating random mutations or making specific changes to the genome.

Once a mutagenesis method is identified, a transformation or transfection is performed 340 by introducing the mutated DNA into a host organism's cell(s). Transformation (in bacteria) or transfection (in eukaryotic cells) allows the mutated DNA to be incorporated into the genome or maintained as an extrachromosomal element. As a result, the mutation is propagated by the cell(s) and expressed as altered genes in the organism. In certain embodiments, this step provides validation of the phenotype (i.e., did the mutagenesis have an effect).

After transformation or transfection, a screening and selection process of mutants is performed 350 according to various embodiments of the invention. In particular, cells or organisms that have successfully incorporated a desired mutation(s) are identified and isolated. One skilled in the art will recognize that a variety of screen methods may be performed within this process. For example, a screening method may be used depending on the type of mutation and organism, including antibiotic resistance, phenotypic changes, or molecular techniques like PCR. In certain instances, this effective screening ensures that only those cells with the desired mutations are further analyzed, saving time and resources.

Upon completion of at least one transformation or transfection, a process of validating the selected mutant(s) occurs 360 according to various embodiments of the invention. This validation process confirms the presence and/or nature of the mutations to support that the observed effects are due to corresponding intended genetic changes. For example, techniques such as sequencing, PCR, or protein analysis verify that the mutations are present and characterize their impact on the gene and protein structure. Oftentimes, this validation ensures the reliability of subsequent analyses.

After the validation process, a functional analysis is performed 370 according to various embodiments of the invention. An assessment of the impact of the mutations on the organism's phenotype, biochemical pathways, or overall fitness provides insights into the gene's function and its role in biological processes. This functional analysis may comprise a range of experiments, from observing growth and development to measuring specific biochemical activities.

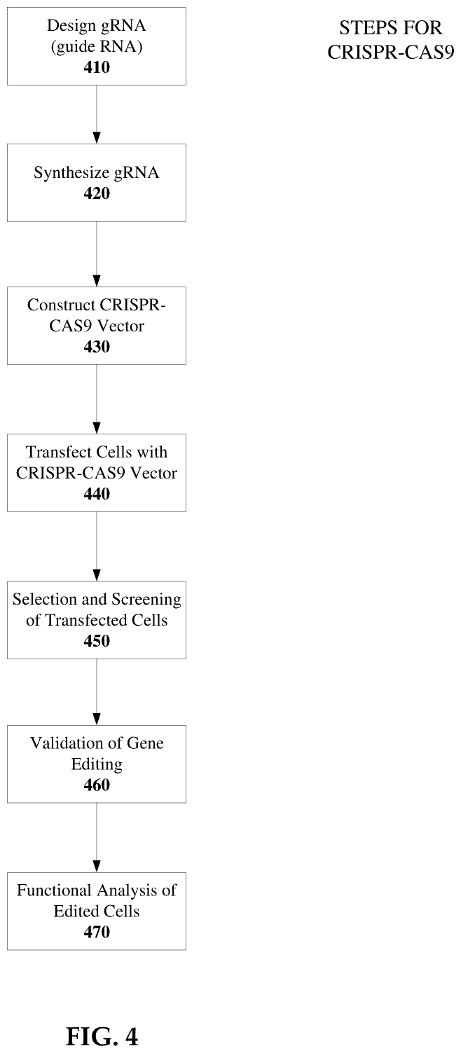

FIG. 4 is a schematic diagram of exemplary steps for performing CRISPR-Cas according to various embodiments of the invention. This process provides targeted genetic modifications to affect gibberellic acid biosynthesis in certain embodiments. As shown, guide RNA (“gRNA) is designed 410 to enable subsequent modification relative to a target DNA sequence. In certain examples, the gRNA determines the target site within the genome where the CRISPR-Cas9 complex will make a cut. The gRNA is designed to ensure specificity to the target DNA sequence, minimizing off-target effects that can lead to unintended mutations. Effectively, this step guides RNAs to genes within the GA pathway to recruit endonucleases to the genes.

After the gRNA is designed, the gRNA is subsequently synthesized 420 according to various embodiments of the invention. In certain examples, the gRNA is synthesized chemically while in other examples the gRNA through in vitro transcription. Accordingly, the genes expressing RNAs designed in step 410 are constructed synthetically. This synthesized gRNA facilitates the CRISPR-Cas9 system to perform binding and cutting the correct DNA sequence.

Upon completion the synthesized gRNA, a CRISPR-Cas9 vector(s) is constructed 430 and correlated to the synthesized gRNA according to various embodiments of the invention. The CRISPR-Cas9 system uses a delivery vehicle to introduce the gRNA and Cas9 protein into target cells. Constructing a CRISPR-Cas9 vector, which may include a plasmid carrying the gRNA and Cas9 gene in certain examples, ensures efficient delivery and expression within the cells. This enables appropriate transformation constructs that could incorporate DNA from step 420 to express gRNAs.

Cells are then transfected 440 with the CRISP-Cas9 vector(s) according to various embodiments of the invention. In certain examples, transfection involves introducing the CRISPR-Cas9 vector into the target cells. This step enables the CRISPR-Cas9 components to enter the cells and reach the nucleus, where they can interact with the genome. In certain examples, the cells or tissue are transformed and potentially selected using antibiotic or herbicide resistance. One skilled in the art will recognize that efficient transfection methods increase the likelihood of successful gene editing within the process.

In many instances, not all the cells will have taken up the CRISPR-Cas9 vector after transfection. Accordingly, a screening and selection process 450 is performed to identify and isolate the cells that have been successfully transfected according to various embodiments of the invention. In certain examples, this process may involve antibiotic selection markers or fluorescent reporters. In embodiments, PCR or sequencing could be used to assay if target genes in a GA pathway are affected. One skilled in the art will recognize that this screening further improves the likelihood that subsequent analyses are performed on cells that contain the CRISPR-Cas9 components.

After identifying and isolating appropriate cells, a validation process occurs 460 on the isolated cells according to various embodiments of the invention. In certain examples, this gene editing validation confirms that the desired modifications have been accurately introduced into the genome. Techniques such as PCR, sequencing, and Western blotting are used to confirm the presence and integrity of the edited gene. Additionally, the phenotype of edited material may be assayed. One skilled in the art will recognize that this step essentially verifies that the gene editing was successful and to assess the accuracy and efficiency of the modifications.

A functional analysis is performed 470 after validation according to various embodiments of the invention. In certain examples, functional analysis involves studying the biological effects of the gene edits on the cells including, but not limited to, observing changes in phenotype, protein expression, cellular behavior, biochemical pathways or other analyses known to one of skill in the art. This functional analysis provides insights into the role of the edited gene and helps to understand the broader implications of genetic modifications

While various embodiments have been described above, they have been presented by way of example only, and not limitation. The descriptions are not intended to limit the scope of the invention to the forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

What is claimed is:

1. A mutagenesis process comprising:
selecting a target gene having a function that is relevant to the mutagenesis process;
extracting DNA from an organism for further processing;
identifying a mutagenesis method based at least in part on the DNA extracted from the organism, the mutagenesis method resulting in at least one mutation to the DNA;
introducing the mutated DNA into a host organism's cell or cells;
confirming the presence and/or nature of the at least one mutation that shows observed effects are due to corresponding intended genetic changes to the mutated DNA;
assessing an impact of the at least one mutation on one or more of an organism's phenotype, biochemical pathway, or overall fitness; and
wherein the impact relates to an increase in cannabis females within the organism.

2. The mutagenesis process of claim 1 further comprising a step of treating the cannabis females with exogenous compounds.

3. The mutagenesis process of claim 2 wherein the exogenous compounds reduce the amount of gibberellic acid produced.

4. The mutagenesis process of claim 1 wherein the organism is altered, through mutagenesis, CRISPR, or other molecular genetics tools, to produce less gibberellic acid.

5. The mutagenesis process of claim 1 wherein the organism is altered, through mutagenesis, CRISPR, or other molecular genetics tools, to be less sensitive to gibberellic acid.

6. The mutagenesis process of claim 1 wherein the mutated DNA is introduced into the host organism's cell or cells using a transformation process.

7. The mutagenesis process of claim 1 wherein the mutated DNA is introduced into the host organism's cell or cells using a transfection process.

8. The mutagenesis process of claim 1 wherein this mutagenesis method may be chemical, physical, biological or a combination thereof.

9. The mutagenesis process of claim 8 wherein the identification of the mutagenesis method correlates to a generation of desired mutations.

10. The mutagenesis process of claim 9 wherein the mutagenesis method uses chemical mutagens that introduce random mutations by altering DNA bases.

11. The mutagenesis process of claim 9 wherein the mutagenesis method uses physical methods that cause DNA damage that leads to mutations during repair.

12. The mutagenesis process of claim 9 wherein the mutagenesis method uses biological methods that provide targeted modifications in a target gene.

13. A method for CRISPR-Cas9 gene editing within a plurality of cannabis cells, the method comprising:
designing a guide RNA (“gNRA”) by determining a target site within a cannabis cell for modification, the cannabis cell being within the plurality of cannabis cells;
synthesizing the gNRA using at least one of a synthesized chemical process or an in vitro transcription;
constructing a CRISPR-Cas9 vector that is correlated to the synthesized gRNA;
transfecting the plurality of cannabis cells using the CRISPR-Cas9 vector;
identifying a subset of cannabis cells that have been successfully transfected by the CRISPR-Cas9 vector; and
performing a functional analysis on the subset of cannabis cells by analyzing biological effects of gene edits on the subset of cannabis cells.

14. The method of claim 13 wherein the functional analysis includes at least one of observing changes in phenotype, protein expression, cellular behavior or biochemical pathways.

15. The method of claim 13 wherein the target site within the cannabis cell is determined to recruit endonucleases to genes within the cannabis cell.

16. The method of claim 13 wherein the synthesized gRNA facilitates a CRISPR-Cas9 system to perform binding and cutting of a correct DNA sequence.

17. The method of claim 13 wherein the CRISPR-Cas9 vector comprises a plasmid carrying the synthesized gRNA and Cas9 gene.

18. The method of claim 13 wherein the step of transfecting the plurality of cannabis cells comprises an introduction of CRISPR-Cas9 components entering the plurality of cannabis cells to reach nuclei within the plurality of cannabis cells.

19. The method of claim 13 wherein the subset of cannabis cells is identified using at least one of antibiotic selection markers and fluorescent reporters.

20. The method of claim 13 wherein at least one of PCR, sequencing, and Western blotting are used to confirm the presence and integrity of an edited gene.
